(19) United States
(12) Patent Application Publication
(10) Pub. No.: US 2025/0332167 A1
(43) Pub. Date: Oct. 30, 2025

(54) PHARMACEUTICAL USE OF CDK 4/6 INHIBITOR

(71) Applicant: Gan & Lee Pharmaceuticals Co., Ltd., Beijing (CN)

(72) Inventors: Lei YIN, Beijing (CN); Zhenglin YAO, Beijing (CN)

(21) Appl. No.: 18/561,161

(22) PCT Filed: May 13, 2022
(86) PCT No.: PCT/CN2022/092706

§ 371 Date: May 9, 2024

(30) Foreign Application Priority Data
May 17, 2021 (CN) 202110534646.8
May 18, 2021 (CN) 202110542586.4
Sep. 14, 2021 (CN) 202111075067.8

(51) Int. Cl. A61K 31/506; A61P 35/00

(52) U.S. Cl. CPC A61K 31/506; A61P 35/00

(57) ABSTRACT

Provided are a method for treating a cancer by a CDK4/6 inhibitor and a corresponding pharmaceutical use. The cancer is glioblastoma or non-small cell lung cancer, and the inhibitor shows a better tumor suppression effect compared with positive control drugs Palbociclib and Abemaciclib.

### FIELD OF THE INVENTION

The present application belongs to the field of medicine and relates to the medical use of a CDK4/6 inhibitor.

### BACKGROUND OF THE INVENTION

Cancer is a major public health concern in many regions of the world. Among them, gliomas are tumors that originate from glial cells, also known as neuroglial tumors. They are the most common primary intracranial tumors. The WHO classification of central nervous system tumors categorizes gliomas into WHO grades I-IV, with grades I and II being low-grade gliomas and grades III and IV being high-grade gliomas. Glioblastoma (GBM) is the most common primary malignant brain tumor in adults, accounting for 54% of gliomas. GBM is the most lethal brain tumor, with only one-third of patients surviving for one year, and less than 5% surviving beyond five years. The treatment of glioblastoma mainly involves surgical removal of the tumor, combined with comprehensive therapies such as radiotherapy and chemotherapy. Major chemotherapy drugs include temozolomide, nitrosoureas, procarbazine, platinum agents, vinca alkaloids, and taxanes. Among them, post-surgery concurrent temozolomide, radiotherapy, and adjuvant chemotherapy have become the standard treatment regimen for newly diagnosed GBM.

Due to its high incidence and mortality rates, lung cancer has become a leading cause of cancer-related deaths worldwide. Lung cancer is a common cause of cancer-related deaths in males and ranks second only to breast cancer in females. In recent years, the incidence and mortality of lung cancer in China have rapidly increased. Currently, based on the biological characteristics and treatment prognosis of lung cancer, the World Health Organization (WHO) classifies it into two major categories: non-small cell lung cancer (NSCLC) and small cell lung cancer (SCLC). NSCLC includes squamous cell carcinoma, adenocarcinoma, and large cell carcinoma. Compared to SCLC, the growth and division of cancer cells in NSCLC are slower, and metastasis occurs relatively later. NSCLC accounts for about 80% of all lung cancers, and approximately 75% of patients are already in the middle to late stages when diagnosed, resulting in a low 5-year survival rate.

5-fluoro-4-(7′-fluoro-2′-methylspiro[cyclopentane-1,3′-indol]-5′-yl)-N-(5-(1-methylpiperidin-4-yl)pyridin-2-yl)pyrimidine-2-amine is a highly selective CDK4/6 inhibitor. The structural formula of the compound is:

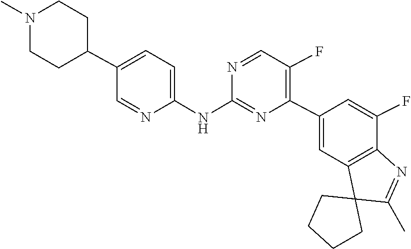 [SMILES: CC1=Nc2c(F)cc(-c3nc(Nc4ccc(C5CCN(C)CC5)cn4)ncc3F)cc2C12CCCC2]

The specific synthesis method is described in detail in WO2017/092635A1.

Therefore, there remains a need to identify drugs with significant efficacy for treating glioblastoma and non-small cell lung cancer.

### SUMMARY OF THE INVENTION

Aiming at the shortcomings of the prior art and the practical demands, the present invention provides a new medical use for 5-fluoro-4-(7′-fluoro-2′-methylspiro[cyclopentane-1,3′-indol]-5′-yl)-N-(5-(1-methylpiperidin-4-yl)pyridin-2-yl)pyrimidine-2-amine.

In one aspect, the present invention provides a method for treating cancer, comprising administering to a subject in need thereof a therapeutically effective amount of the compound (I) or a pharmaceutically acceptable salt thereof,

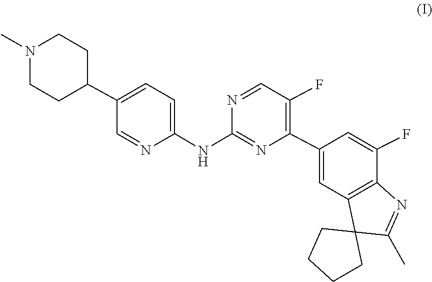 [SMILES: CC1=Nc2c(F)cc(-c3nc(Nc4ccc(C5CCN(C)CC5)cn4)ncc3F)cc2C12CCCC2]

In one embodiment, the pharmaceutically acceptable salt of the compound (I) is the fumarate salt of the compound (I).

In another aspect, the present invention provides use of the compound of formula (I) or a pharmaceutically acceptable salt thereof in the preparation of a medicament for treating cancer, wherein the cancer is glioblastoma or non-small cell lung cancer.

In one embodiment, the pharmaceutically acceptable salt of the compound (I) is the fumarate salt of the compound (I).

In a further aspect, the present invention provides the compound of formula (I) or a pharmaceutically acceptable salt thereof, for use in treating cancer, wherein the cancer is glioblastoma or non-small cell lung cancer.

In one embodiment, the pharmaceutically acceptable salt of the compound (I) is the fumarate salt of the compound (I).

Unless specifically stated, the term “compound of the present invention refers to compound (I) and a salt thereof, including pharmaceutically acceptable salts of the compound and all stereoisomers (including but not limited to diastereomers and enantiomers), tautomers, isotopic compounds, prodrugs thereof, solvates, and hydrates.

The term “pharmaceutically acceptable salt” refers to a salt that retains the biological activity of the free acids and bases of a particular compound without biological adverse effects. Examples of pharmaceutically acceptable salts include but are not limited to salts of compound (I) formed with acids such as hydrochloric acid, hydrobromic acid, sulfuric acid, nitric acid, phosphoric acid, acetic acid, trifluoroacetic acid, propionic acid, hexanoic acid, heptanoic acid, cyclopentanepropionic acid, isobutyric acid, pyruvic acid, lactic acid, malonic acid, succinic acid, tartaric acid, citric acid, benzoic acid, cinnamic acid, mandelic acid, methanesulfonic acid, ethanesulfonic acid, 1,2-ethanedisulfonic acid, 2-hydroxyethanesulfonic acid, benzenesulfonic acid, para-chlorobenzenesulfonic acid, para-toluenesulfonic acid, 3-phenylpropionic acid, trimethylacetic acid, t-butylacetic acid, dodecylsulfuric acid, glucuronic acid, glutamic acid, hydroxynaphthoic acid, salicylic acid, and stearic acid; preferred fumaric acid.

The terms “treatment”, “treat” and “treating” generally refer to obtaining desired pharmacological and/or physiological effects. These effects may involve partial or complete stabilization or cure of a disease and/or side effects resulting from the disease, and can be therapeutic. The terms “treatment”, “treat” and “treating” as used herein encompasse any treatment for a patient's disease, including: (a) preventing symptoms of a disease, i.e., preventing the progression of the disease; or (b) alleviating symptoms of a disease, i.e., causing regression of the disease or symptoms.

The term “subject” refers to mammals, preferably humans.

### Advantages of the Present Invention

1) The compounds of the present invention exhibit significant inhibitory effects on glioblastoma, and when compared to the positive control drugs Palbociclib and Abemaciclib, demonstrate superior tumor inhibition effects.
2) The compounds of the present invention exhibit significant inhibitory effects on non-small cell lung cancer, and when compared to the positive control drugs Palbociclib and Abemaciclib, demonstrate superior tumor inhibition effects.
3) The compounds of the present invention do not exhibit significant drug toxicities or adverse effects, and show good tolerability.

### BRIEF DESCRIPTION OF THE DRAWINGS

FIG. 1: The body weight changes of each treatment group in a xenograft mouse model of human glioblastoma.

FIG. 2: The body weight changes of each treatment group in a xenograft mouse model of human non-small cell lung cancer NCI-H2228.

FIG. 3: The body weight changes of each treatment group in a xenograft mouse model of human non-small cell lung cancer NCI-H1975.

### DETAILED DESCRIPTION OF THE INVENTION

The experimental methods in the following examples, unless otherwise specified, are conventional methods. The chemical raw materials, reagents, etc. and used in the following examples, unless otherwise specified, are commercially available products.

### Abbreviations

DLT: Dose-Limiting Toxicity
DAPI: 4′,6-Diamidino-2-phenylindole
DMSO: Dimethyl Sulfoxide
FBS: Fetal Bovine Serum
MEM: Minimum Essential Medium
NEAA: Non-Essential Amino Acids
HEC: Hydroxyethyl Cellulose
HPC: Hydroxypropyl Cellulose
PBS: Phosphate-Buffered Saline
RTV: Relative Tumor Volume
SEM: Standard Error of the Mean
T/C: Relative tumor growth rate
TGI: Relative tumor inhibition rate
TV: Tumor Volume
Vehicle: Solvent

### EXAMPLE 1

Fumarate salt of 5-fluoro-4-(7′-fluoro-2′-methylspiro[cyclopentane-1,3′-indol]-5′-yl)-N-(5-(1-methylpiperidin-4-yl)pyridin-2-yl)pyrimidine-2-amine (Compound A)

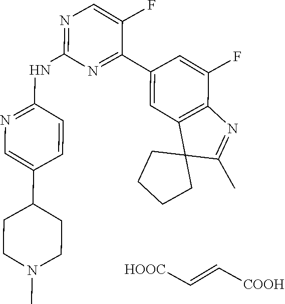 [SMILES: CC1=Nc2c(F)cc(-c3nc(Nc4ccc(C5CCN(C)CC5)cn4)ncc3F)cc2C12CCCC2 ; O=C(O)/C=C/C(=O)O]

First, 5-fluoro-4-(7′-fluoro-2′-methylspiro[cyclopentane-1,3′-indol]-5′-yl)-N-(5-(1-methylpiperidin-4-yl)pyridin-2-yl)pyrimidine-2-amine was prepared referring to the synthesis method described in WO2017/092635A1.

Subsequently, under nitrogen protection, at room temperature, dichloromethane (22.0 volumes) and ethanol (22.0 volumes) were added to the reaction vessel, followed by the addition of 5-fluoro-4-(7′-fluoro-2′-methylspiro[cyclopentane-1,3′-indol]-5′-yl)-N-(5-(1-methylpiperidin-4-yl)pyridin-2-yl)pyrimidine-2-amine (2.070 kg) while stirring. The temperature was raised to 30-40 degrees Celsius, stirring until complete dissolution, then cooled down to room temperature, and the solution was transferred to a solvent tank for later use. Under nitrogen protection, the above solution was filtered through a microfiltration filter and transferred to the reaction vessel. The mixture was stirred, and atmospheric pressure was used to evaporate dichloromethane and ethanol. The reaction vessel was then maintained at a temperature of 80±5 degrees Celsius, and an ethanol solution of fumaric acid (1.0 eq) (12 volumes) was slowly added dropwise into the reaction vessel. The mixture was stirred and kept warm overnight. The temperature was lowered to 20-30 degrees Celsius, and stirring was continued for at least 1 hour. The mixture was centrifuged, and the filter cake was collected. The filter cake was placed in a vacuum drying oven and dried overnight to obtain 1.605 kg of fumarate salt of 5-fluoro-4-(7′-fluoro-2′-methylspiro[cyclopentane-1,3′-indol]-5′-yl)-N-(5-(1-methylpiperidin-4-yl)pyridin-2-yl)pyrimidine-2-amine (Compound A) with a yield of 63.6%.

### EXAMPLE 2

### Measurement of Inhibitory Effect of the Compounds of the Present Invention on Proliferation of Human Glioblastoma Cells U118MG

Human glioblastoma cell U118MG was purchased from NANJING COBIOER BIOSCIENCES CO., LTD. The positive control drug Abemaciclib was prepared according to the synthesis method disclosed in WO2010075074A1. The cell detection equipment used was the In Cell Analyzer 2200 (GE Healthcare). The reagents and materials used in the experiment are shown in the following table:

TABLE 1
Reagents and Materials

| Reagents and Materials | Lot No. | Brand |
|---|---|---|
| DMSO | D2650 | Sigma |
| PBS | 20012-027 | Gibco |
| DAPI | D8417 | Sigma |
| Formaldehyde | 47608 | Sigma |
| Triton ™ TM X-100 | T9284 | Sigma |
| MEM | 12561-056 | Gibco |
| MEM NEAA | 2079705 | Gibco |
| Sodium pyruvate | 11140-050 | Gibco |
| FBS | 10099141 | Gibco |
| penicillin/streptomycin | 15140122 | Thermo Fisher |
| 96-well black clear-bottom cell plate. | 6005182 | PE |

Abemaciclib and Compound A were dissolved in DMSO to prepare 10 mM stock solutions, which were then stored at −80° C. for long-term preservation. A 5 μL aliquot of the 10 mM stock solutions of Abemaciclib and Compound A were taken and were diluted into working solution of 60 μM respectively. With 60 μM as the initial concentration, ten points were obtained by serial five-fold dilution.

Medium for U118 MG is the MEM (low glucose) medium supplemented with 10% FBS, 1% penicillin/streptomycin, 1% sodium pyruvate, and 1% MEM NEAA. U118 MG cells were seeded at a density of 4000 cells/100 μL/well in a 96-well black clear-bottom plate and incubated at 37° C. overnight. The plate was then taken, and 20 μL of the prepared diluted samples was added to the wells. The cells were treated at 37° C. for 72 hours. After 72 hours, the plate was taken, and a neutral formaldehyde fixation solution (formaldehyde:PBS=1:9) was added at 50 μL/well. The cells were fixed at room temperature for 10-30 minutes. The wells were washed twice with 1× PBS (100 μL/well), and then permeabilized with 0.2% Triton™-X100 for 5-10 minutes (100 μL/well). After another two washes with 1× PBS (100 μL/well), the cells were stained with DAPI (diluted 1:5000 in PBS) for 20 minutes at room temperature in the dark (50 μL/well). After three more washes with 1× PBS (100 μL/well), 100 μL of 1× PBS was added to each well. The plate was scanned by using an In Cell Analyzer, and the number of cells in each well was analyzed. The inhibition rate of each compound at various concentration points was calculated by using the formula provided. The IC₅₀ values were determined by curve fitting using GraphPad Prism 6.0 software.

$$Inhibition rate at each concentration point ( inh \% ) = ( Cell count at zero point concentration - Cell count at each concentration point ) / Cell count at zero point concentration \times 100 \%$$

The experimental results are shown in Table 2, indicating that Compound A exhibits significant proliferation inhibition activity against the U118MG cells. Compared to the positive control drug Abemaciclib, Compound A demonstrates higher proliferation inhibition activity.

TABLE 2
Inhibitory Activity of Test Compounds
on U118MG Cells Proliferation

| Samples | IC₅₀ (nM) |
|---|---|
| Abemaciclib | 86.0 ± 22.9 |
| Compound A | 23.2 ± 5.2 |

### EXAMPLE 3

### Efficacy Experiment of the Invention Compound in a Human Glioblastoma Xenograft Mouse Model

Human glioblastoma model (BN2289, Crown Bioscience Co., Ltd.) tumor fragments were cut into small pieces with a diameter of 2-3 mm and subcutaneously inoculated into the right side of BALB/C nude mice. Tumor growth was monitored regularly, and when the tumors grew to an average volume of approximately 100-200 mm³, mice were randomly divided into groups based on the tumor size.

The positive control drugs were Palbociclib and Abemaciclib. Palbociclib was prepared by the inventors according to the synthesis method disclosed in WO2003062236A1, and Abemaciclib was prepared by the inventors according to the synthesis method disclosed in WO2010075074A1.

The experiment included groups treated with Compound A at doses of 5.0 mg/kg, 10.0 mg/kg, 25.0 mg/kg, and 50.0 mg/kg, a Palbociclib group at a dose of 25.0 mg/kg, an Abemaciclib group at a dose of 25.0 mg/kg, and a Vehicle group. The preparations of the test compounds and reference compound are shown in Table 3.

TABLE 3
Preparations of Test Compounds and Reference Compound

| | | Concentration |
|---|---|---|
| Samples | Preparation | (mg/ml) |
| Vehicle (0.1% | Weigh 0.5 g of HEC, add 498.75 ml of sterile water to | — |
| HEC, 0.25% | dissolve it thoroughly, then add 1.25 ml of Tween 80 | |
| Tween80) | and mix well. Set aside. | |
| Compound A | Weigh 37.42 mg of Compound A and dissolve it in 6.0 | 5 |
| 50 mg/kg group | ml of vehicle. Mix thoroughly using vortex, resulting in | |
| | a 5.0 mg/ml solution of Compound A. | |
| Compound A | Take 2.5 ml of the 5.0 mg/ml Compound A solution and | 2.5 |
| 25 mg/kg group | add 2.5 ml of vehicle. Mix well using vortex, obtaining | |
| | a 2.5 mg/ml solution of Compound A in a total volume | |
| | of 5.0 ml. | |
| Compound A | Take 2.0 ml of the 2.5 mg/ml Compound A solution and | 1 |
| 10 mg/kg group | add 3.0 ml of vehicle. Mix well using vortex, resulting | |
| | in a 1.0 mg/ml solution of Compound A in a total | |
| | volume of 5.0 ml. | |
| Compound A | Take 1.5 ml of the 1.0 mg/ml Compound A solution and | 0.5 |
| 5 mg/kg group | add 1.5 ml of vehicle. Mix well using vortex, resulting | |
| | in a 0.5 mg/ml solution of Compound A in a total | |
| | volume of 3.0 ml. | |
| Palbociclib | Weigh 11.35 mg of Compound Palbociclib and dissolve | 2.5 |
| 25 mg/kg | it in 3.5 ml of vehicle. Mix well using vortex and set | |
| | aside. | |
| Abemaciclib | Weigh 10.43 mg of Compound Abemaciclib and | 2.5 |
| 25 mg/kg | dissolve it in 3.5 ml of vehicle. Mix well using vortex. | |
| | and set aside. | |

Each group consisted of 8 mice and received oral gavage administration once daily for a total of 28 days. Regular observations were made on tumor volume and changes in body weight. Efficacy evaluation was conducted based on the relative tumor growth rate (T/C) and the relative tumor inhibition rate (TGI).

The formula for calculating tumor volume was: Tumor Volume (mm³)=½×(a×b²) (where “a” represents the long diameter and “b” represents the short diameter).

The relative tumor growth rate (T/C) is the percentage value of the relative tumor volume or weight between the treatment group and the control group at a certain time point. The calculation formula is as follows:
T/C(%)=T_{RTV}/C_{RTV}×100% (T_{RTV}: Average RTV of the treatment group; C_{RTV}: Average RTV of the Vehicle group; RTV=Vₜ/V₀, where V₀ is the tumor volume of the animal at the time of grouping and Vₜ is the tumor volume of the animal after treatment).

The formula for calculating the relative tumor inhibition rate (TGI) is as follows: TGI(%)=(1−T/C)×100%. (T and C are the relative tumor volume (RTV) or weight (TW) of the treatment group and control group at a specific time point, respectively).

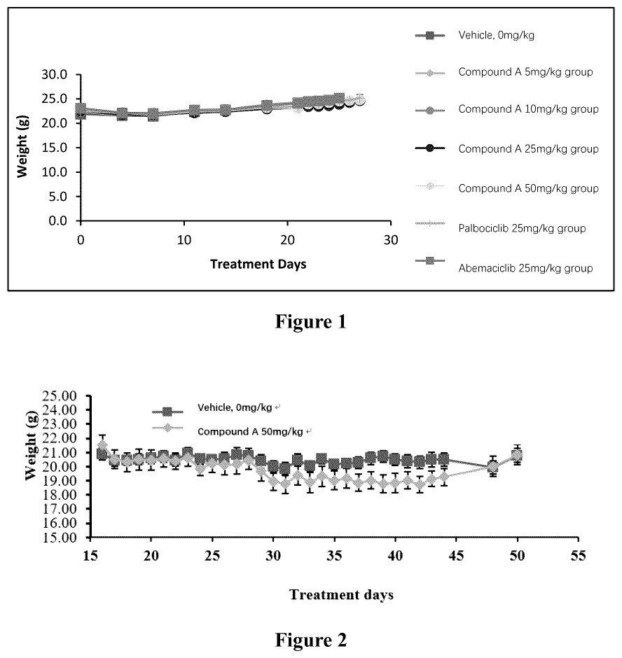

The results are shown in Table 4 and FIG. 1. These results indicate that the treatment groups with Compound A (5 mg/kg, 10 mg/kg, 25 mg/kg, 50 mg/kg) exhibited tumor inhibition effects on the 21st day, with relative tumor growth rates (T/C) of 95.08%, 88.24%, 60.61%, and 43.56%, respectively. Statistical significance was observed compared to the Vehicle group (all p-values<0.001), and a dose-dependent relationship was evident.

The treatment groups with Compound A (25 mg/kg, 50 mg/kg) on the 21st day exhibited relative tumor growth rates (T/C) of 60.61% and 43.56%, respectively, showing better tumor inhibition effects compared to the positive drugs Palbociclib (25 mg/kg) and Abemaciclib (25 mg/kg) groups.

Throughout the experiment, the body weight of animals overall remained stable, and no significant drug toxicity was observed in any of the treatment groups. The test drug demonstrated good tolerance.

From this, it can be seen that Compound A exhibited significant tumor inhibitory effects in the human glioblastoma xenograft mouse model, and the compound showed no significant toxic side effects, indicating promising clinical prospects for the treatment of glioblastoma.

TABLE 4
Inhibitory Activity of Test Compounds in the Glioblastoma Xenograft Model

| | After 21 days of administration | | | | | |
|---|---|---|---|---|---|---|
| | | | Relative | | | |
| | Day 0 Tumor | | Average | | | |
| Experimental | Volume | Day 21Tumor | Tumor | T/C | TGI | |
| Group | (mm³) | Volume (mm³) | Volume | (%) | (%) | P value |
| Vehicle group | 150.31 ± 9.37 | 2201.25 ± 225.53 | 14.51 | — | — | — |
| Compound A | 149.98 ± 10.72 | 2038.41 ± 177.46 | 13.80 | 95.08 | 4.92 | P < 0.001 |
| 5 mg/kg group | | | | | | |
| Compound A | 150.11 ± 10.34 | 1900.70 ± 134.20 | 12.81 | 88.24 | 11.76 | P < 0.001 |
| 10 mg/kg group | | | | | | |
| Compound A | 150.19 ± 10.05 | 1302.81 ± 119.99 | 8.80 | 60.61 | 39.39 | P < 0.001 |
| 25 mg/kg group | | | | | | |
| Compound A | 150.35 ± 11.74 | 938.13 ± 52.26 | 6.32 | 43.56 | 56.44 | P < 0.001 |
| 50 mg/kg group | | | | | | |
| Palbociclib | 149.93 ± 9.84 | 1417.98 ± 121.86 | 9.58 | 66.01 | 33.99 | P < 0.001 |
| 25 mg/kg | | | | | | |
| Abemaciclib | 149.97 ± 9.55 | 1627.31 ± 100.66 | 10.90 | 75.13 | 24.87 | P < 0.001 |
| 25 mg/kg | | | | | | |

Note:
The P-value was calculated using a t-test to compare each group with the Vehicle group.

### EXAMPLE 4

### Inhibitory Proliferation Assay of the Present Invention Compound on Non-Small Cell Lung Cancer Cell Lines

Sixteen non-small cell lung cancer cell lines, including NCI-H1792, NCI-H1703, NCI-H441, SNU-761, NCI-H1975, NCI-H358, NCI-H1838, NCI-H1915, A549, SK-MES-1, NCI-H292, PC-9, NCI-H460, NCI-H23, NCI-H1581, and HLF, were used for the proliferation inhibition assay of the present invention compound. The positive control drug Abemaciclib was synthesized by the inventors according to the method disclosed in WO2010075074A1. The cell detection equipment used was In Cell Analyzer 2200 (GE Healthcare). The reagents and materials used in the experiment are listed in the following table:

TABLE 5
Reagents and Materials

| Reagents and Materials | Lot No. | Brand |
|---|---|---|
| DMSO | D2650 | Sigma |
| PBS | 20012-027 | Gibco |
| DAPI | D8417 | Sigma |
| Formaldehyde | 47608 | Sigma |
| Triton ™ X-100 | T9284 | Sigma |
| 96-well black transparent-bottom cell plate | 6005182 | PE |

Abemaciclib and Compound A were dissolved in DMSO to prepare 10 mM stock solutions, which were stored at −80° C. in a freezer for long-term preservation. A 5 μL aliquot of the 10 mM stock solutions of Abemaciclib and Compound A was taken and further diluted to prepare working solutions of 60 μM. With 60 μM as the initial concentration, ten points were obtained by serial five-fold dilution.

Each kind of cell in logarithmic growth phase was inoculated with 4000 cells/100 μl/well into a black transparent bottom 96-well plate and cultured overnight at 37° C. The next day, the plate was taken out, and 20 μL of the prepared sample dilutions was added to each well. The plate was then subjected to treatment at 37° C. for 72 hours. After the treatment period of 72 hours, the plate was taken out and 50 μL of neutral formaldehyde fixative (formaldehyde: PBS=1:9, 50 μl/well) was added to each well. The plate was then fixed at room temperature for 10-30 minutes. Subsequently, the wells were washed twice with 1× PBS (100 μl/well) and treated with 0.2% Triton™-X100 for 5-10 minutes to permeabilize the cells. After the permeabilization step, the cells were washed twice with 1× PBS (100 μl/well) and then subjected to DAPI staining by adding 50 μL of a diluted DAPI solution (PBS 1:5000 dilution) to each well. The plate was incubated at room temperature in the dark for 20 minutes during the staining process. Following DAPI staining, the wells were washed three times with 100 μL of 1× PBS (100 μl/well). Finally, 100 μL of 1× PBS was added to each well. The plate was then scanned using an In Cell Analyzer to analyze the number of cells per well. The inhibitory rates of each compound at different concentration points were calculated by using the following formula, and IC₅₀ values were obtained through curve fitting using GraphPad Prism 6.0 software.

$$Inhibition rate at each concentration point ( inh \% ) = ( Cell count at zero point concentration - Cell count at each concentration point ) / Cell count at zero point concentration \times 100 \%$$

The experimental results are presented in Table 2. These results demonstrate that Compound A exhibits significant proliferation-inhibiting activity on all 16 tested non-small cell lung cancer cell lines. In comparison to the positive control drug Abemaciclib, Compound A shows higher proliferation inhibition activity.

TABLE 6
Inhibitory Activity of Test Compounds on Proliferation
of Various Non-Small Cell Lung Cancer Cell Lines

Samples IC₅₀ (μM)

| Cell Line | Compound A | Abemaciclib |
|---|---|---|
| NCI-H1792 | 0.003 | 0.023 |
| NCI-H1703 | 0.003 | 0.093 |
| NCI-H441 | 0.007 | 0.04 |
| SNU-761 | 0.008 | 0.054 |
| NCI-H1975 | 0.01 | 0.112 |
| NCI-H358 | 0.017 | 0.044 |
| NCI-H1838 | 0.0479 | 0.0902 |
| NCI-H1915 | 0.053 | 0.324 |
| A549 | 0.066 | 0.104 |
| SK-MES-1 | 0.067 | 0.148 |
| NCI-H292 | 0.073 | 0.149 |
| PC-9 | 0.18 | 0.457 |
| NCI-H460 | 0.235 | 0.654 |
| NCI-H23 | 0.243 | 0.904 |
| NCI-H1581 | 1.116 | 3.231 |
| HLF | 2.014 | 4.948 |

### EXAMPLE 5

### Pharmacodynamic Experiment of Present Invention Compounds in Human Non-Small Cell Lung Cancer NCI-H2228 Xenograft Mouse Model

Human non-small cell lung cancer cells NCI-H2228 were cultured in RPMI-1640 medium containing 10% fetal bovine serum. Log-phase NCI-H2228 cells were collected, suspended in PBS to an appropriate concentration, and mixed with extracellular matrix gel in a 1:1 ratio for subcutaneous tumor implantation in mice. Twenty female mice were subcutaneously implanted with NCI-H2228 cells on the right side. The cells were suspended in PBS and mixed evenly with matrix gel in a 1:1 ratio (0.1 ml per mouse). When the average tumor volume reached 143 mm³, the mice were randomly divided into groups based on tumor size. The groups received either the solvent or Compound A from Example 1 (50 mg/kg), administrated by oral intragastric administration once a day for 28 days. Tumor volume and body weight changes were regularly observed. All animals were euthanized on day 50 after cell inoculation. The tumors were weighed and photographed. The calculation methods for tumor volume, relative tumor growth rate (T/C), and tumor growth inhibition rate (TGI) were shown in Example 3.

The results are presented in Table 7 and FIG. 2 which indicate that the average tumor volume of mice in the Vehicle group and Compound A (50 mg/kg) treatment group on the 50th day after cell implantation were 766.64 mm³ and 506.53 mm³, respectively. Compared to the Vehicle group, the treatment group exhibited significant inhibition against NCI-H2228 human non-small cell lung cancer (p=0.003), with a relative tumor growth inhibition rate (TGI) of 36%. Most mice in the treatment group experienced slight weight loss, but overall, they exhibited good tolerability during the treatment period.

It can be seen that Compound A from Example 1 demonstrates significant tumor inhibition in the human non-small cell lung cancer NCI-H2228 xenograft mouse model, and the compound shows no obvious adverse effects, indicating promising clinical potential for treating non-small cell lung cancer.

TABLE 7
Inhibitory Activity of Test Compounds in Treating NCI-
H2228 Xenograft Model of Non-Small Cell Lung Cancer

| | | Day 50 post-implantation | | | | |
|---|---|---|---|---|---|---|
| | | | Tumor | | | |
| | | Tumor volume | relative | | | |
| | Animal | (mm³) | volume | | | P |
| Groups | number | (x̅ ± SEM) | (x̅ ± SEM) | TGI(%) | T/C (%) | value |
| Vehicle | 10 | 766.64 ± 60.06 | 5.26 ± 0.50 | — | — | — |
| CompoundA | 10 | 506.53 ± 44.73 | 3.38 ± 0.23 | 36 | 64 | 0.003 |
| 50 mg/kg | | | | | | |
| group | | | | | | |

Note:
P-values were calculated using the T-test to compare each group with the Vehicle group.

### EXAMPLE 6

### Pharmacodynamic Experiment of Present Invention Compound in Human Non-Small Cell Lung Cancer NCI-H1975 Xenograft Mouse Model

NCI-H1975 cells were cultured in RPMI1640 medium containing 10% fetal bovine serum. Log-phase NCI-H1975 cells were collected, suspended in PBS to an appropriate concentration, and used for subcutaneous tumor implantation in Balb/c nude mice. Each female mouse was implanted with 0.1 ml of NCI-H1975 cells subcutaneously. When the average tumor volume reached 171 mm³, the mice were randomly divided into groups based on tumor size.

The positive control drugs were Palbociclib and Abemaciclib. Palbociclib was synthesized according to the method described in WO2003062236A1, and Abemaciclib was synthesized according to the method described in WO2010075074A1.

The experiment included Compound A at doses of 5.0 mg/kg, 10.0 mg/kg, 25.0 mg/kg, and 50.0 mg/kg, Palbociclib at 25.0 mg/kg, Abemaciclib at 25.0 mg/kg, and the Vehicle group. The preparation of test compounds and control compound are shown in Table 8.

TABLE 8
Preparation of Test Compounds and Control compound

| | | Concentration |
|---|---|---|
| Samples | Preparation | (mg/ml) |
| Vehicle(0.1% | Weigh 0.5 g of HPC powder, add 500 ml of sterile water, stir | — |
| HPC, 0.25% | to dissolve. After complete dissolution, add 1.25 ml of | |
| Tween80) | Tween80, and mix by vortex to obtain the solution. | |
| Compound A | Weigh 29.76 mg of compound A powder, add 4.8 ml of | 5 |
| 50 mg/kg group | Vehicle, mix by vortex and ultrasonication, to obtain a | |
| | 5 mg/ml solution of compound A. | |
| Compound A | Transfer 2.4 ml of the 5 mg/ml compound A solution, add | 2.5 |
| 25 mg/kg group | 2.4 ml of Vehicle, mix by vortex and ultrasonication, to | |
| | obtain a 2.5 mg/ml solution of compound A. | |
| Compound A | Transfer 1.92 ml of the 2.5 mg/ml compound A solution, | 1 |
| 10 mg/kg group | add 2.88 ml of Vehicle, mix by vortex and ultrasonication, | |
| | to obtain a 1 mg/ml solution of compound A. | |
| Compound A | Transfer 2.4 ml of the 1 mg/ml compound A solution, add | 0.5 |
| 5 mg/kg group | 2.4 ml of Vehicle, mix by vortex and ultrasonication, to | |
| | obtain a 0.5 mg/ml solution of compound A. | |
| Palbociclib | Weigh 7.68 mg of Palbociclib powder, add 2.4 ml of | 2.5 |
| 25 mg/kg group | Vehicle, mix by vortex and ultrasonication. | |
| Abemaciclib | Weigh 7.14 mg of Abemaciclib powder, add 2.4 ml of | 2.5 |
| 25 mg/kg group | Vehicle, mix by vortex and ultrasonication. | |

Each group consisted of 8 mice, and oral gavage administration was performed once daily for a total of 21 days. Tumor volume and body weight changes were observed regularly, and efficacy evaluation was conducted based on relative tumor proliferation rate (T/C) and relative tumor growth inhibition rate (TGI). The calculation methods for tumor volume, T/C, and TGI are described in Example 3.

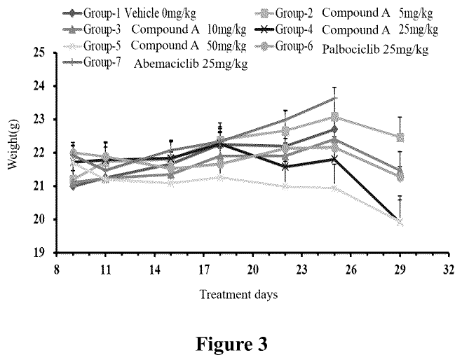

The results are presented in Table 9 and FIG. 3. These results demonstrated that on Day 16 of treatment initiation, Compound A at doses of 25 mg/kg and 50 mg/kg exhibited highly significant anti-tumor effects (p-values<0.001), with TGI values of 50% and 69%, and average tumor volumes of 1141 mm³ and 709 mm³, respectively. At a dose of 10 mg/kg, statistically significant anti-tumor effects were also observed (p=0.025), with a TGI of 28% and average tumor volume of 1647 mm³. A slight anti-tumor effect was observed at a dose of 5 mg/kg (p=0.333), with a TGI of 15% and average tumor volume of 1889 mm³. Compound A exhibited a good dose-effect relationship.

The positive control drug Palbociclib at 25 mg/kg also showed a statistically significant difference in anti-tumor effect (p=0.002), with a TGI of 34% and average tumor volume of 1469 mm³. Compound A treatment groups (25 mg/kg and 50 mg/kg) outperformed the positive control drug Palbociclib (25 mg/kg) in tumor inhibition. The positive control drug Abemaciclib at 25 mg/kg did not exhibit significant anti-tumor effects (p=0.911), with a TGI of 7% and average tumor volume of 2071 mm³. Compound A treatment groups showed better tumor inhibition than the positive control drug Abemaciclib (25 mg/kg).

Throughout the experiment, no decrease in body weight was observed in any treatment group until Day 16 of treatment initiation. On Day 20 of treatment initiation, a slight decrease in body weight was observed in the Compound A groups at doses of 25 mg/kg and 50 mg/kg, as well as the positive control drug Palbociclib group. Overall, the experimental mice demonstrated good tolerance to the test compounds.

It can be seen that Compound A exhibited significant anti-tumor effects in the human non-small cell lung cancer NCI-H1975 xenograft mouse model, and the compound showed noobvious adverse effects, indicating promising clinical prospects for the treatment of non-small cell lung cancer.

TABLE 9
Inhibition Activity of Test Compounds in Treating NCI-
H1975 Xenograft Mouse Model of Non-Small Cell Lung Cancer

| | 16 days after administration | | | | |
|---|---|---|---|---|---|
| | Tumor | | | | |
| Experimental | volume | Tumor relative | | T/C | |
| Group | (x̅ ± S) | volume (x̅ ± S) | TGI | (%) | P value |
| Negative Control | 2237 ± 141 | 11.6 ± 0.9 | — | — | — |
| Compound A | 1889 ± 60 | 9.8 ± 0.6 | 15 | 85 | 0.333 |
| 5 mg/kg group | | | | | |
| Compound A | 1647 ± 243 | 8.4 ± 1.0 | 28 | 72 | 0.025 |
| 10 mg/kg group | | | | | |
| Compound A | 1141 ± 112 | 5.8 ± 0.3 | 50 | 50 | <0.001 |
| 25 mg/kg group | | | | | |
| Compound A | 709 ± 47 | 3.6 ± 0.2 | 69 | 31 | <0.001 |
| 50 mg/kg group | | | | | |
| Palbociclib | 1469 ± 146 | 7.7 ± 1.0 | 34 | 66 | 0.002 |
| 25 mg/kg group | | | | | |
| Abemaciclib | 2071 ± 148 | 10.8 ± 1.0 | 7 | 93 | 0.911 |
| 25 mg/kg group | | | | | |

Note:
P-values were determined using a T-test to compare each group with the Vehicle group.

The present invention has been described through the above examples. However, it should be understood that the above examples are provided for illustrative purposes and are not intended to limit the scope of the present invention to the described embodiments. Additionally, those skilled in the art will appreciate that the present invention is not limited to the above examples. Based on the teachings of the present invention, various modifications and alterations can be made, all of which fall within the scope of protection as required by the appended claims and their equivalents.

1-2. (canceled)

3. A method for treating cancer, comprising administering to a subject in need thereof a therapeutically effective amount of a compound of formula (I) or a pharmaceutically acceptable salt thereof, 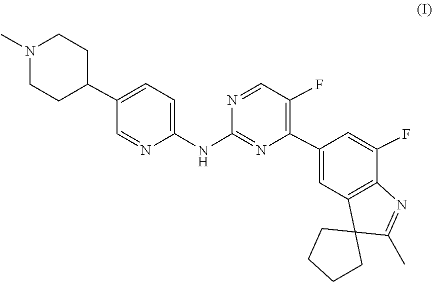 [SMILES: CC1=Nc2c(F)cc(-c3nc(Nc4ccc(C5CCN(C)CC5)cn4)ncc3F)cc2C12CCCC2]
wherein the cancer is glioblastoma or non-small cell lung cancer.

4. The method according to claim 3, characterized in that, wherein the pharmaceutically acceptable salt of the compound of formula (I) is the fumarate salt of the compound of formula (I).

5-6. (canceled)

7. A pharmaceutical composition comprising a fumarate salt of a compound of formula (I), 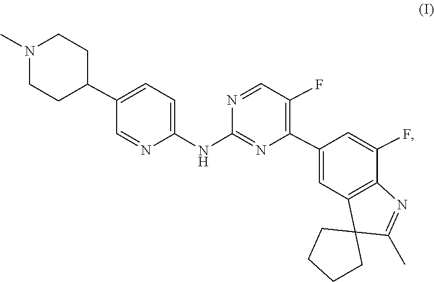 [SMILES: CC1=Nc2c(F)cc(-c3nc(Nc4ccc(C5CCN(C)CC5)cn4)ncc3F)cc2C12CCCC2]
and one or more pharmaceutically acceptable excipients.

8. A method for treating cancer, comprising administering to a subject in need thereof a therapeutically effective amount of the pharmaceutical composition according to claim 7.
